(19) United States
(12) Patent Application Publication
(10) Pub. No.: US 2019/0374449 A1
(43) Pub. Date: Dec. 12, 2019

(54) METALLIC GEL NAIL POLISH AND METHOD OF APPLICATION

(71) Applicants: Benny Dadon, Chatsworth, CA (US); Lior Dadon, Chatsworth, CA (US); BTD Imports, Inc. dba Joya Mia, Chatsworth, CA (US)

(72) Inventors: Jacky Zhao, Changde City (CN); Lior Dadon, Chatsworth, CA (US); Benny Dadon, Chatsworth, CA (US)

(73) Assignee: BTD Imports, Inc. dba Joya Mia, Chatsworth, CA (US)

(21) Appl. No.: 16/120,186

(22) Filed: Aug. 31, 2018

Related U.S. Application Data
(60) Provisional application No. 62/683,601, filed on Jun. 11, 2018.

(51) Int. Cl. A61K 8/26; A61Q 3/02; A61K 8/04; A61K 8/49; A61K 8/81; A61K 8/34; A61K 8/37; A61K 8/35

(52) U.S. Cl. CPC A61K 8/26; A61Q 3/02; A61K 8/042; A61K 8/4973; A61K 8/8147; A61K 8/34; A45D 29/00; A61K 8/35; A61K 2800/94; A61K 2800/26; A61K 2800/81; A61K 2800/30; A61K 8/37

(57) ABSTRACT

The present invention is a metallic gel nail polish composition and method of application. The polish lacks harmful airborne toxins and is comprised of monomers, aluminum, epoxy acrylate oligomer, acrylic acid polymer and photoinitiator. The aluminum is deduced to a semi-solid phase before mixing in with the remaining chemicals. When the composition is applied to a solid surface, the surface tension between the aluminum and remaining chemicals disperses the aluminum evenly across a surface resulting in a shiny, metallic color. The composition is cured for about 2 minutes using an about 36-watt UV light or about 10-60 seconds using an LED light. The composition may be applied above a cured standard base coat or under a cured standard top coat. If a colored metallic color is desired, a translucent gel polish is applied atop the cured metallic gel polish whereby the aluminum particles reflect off the translucent coat.

### BACKGROUND

Nail polish application is a popular way to decorate fingernails and toenails. Nail polish may contain toxic airborne chemicals such as formaldehyde (a well-known carcinogen), toluene (may cause reproductive harm), and triphenyl phosphate (a suspected endocrine disruptor), and dries as the solvent evaporates, releasing harmful fumes into the air inhaled by persons nearby.

While nail polish comes in a variety of colors, a desired stylized look includes colors having a shiny, metallic finish. Several nail polishes advertise this finish, but the resulting colors are dull and lack the shiny effect. One known way to achieve the desired finish is applying a chrome powder to an underlying coat of nail polish through a time-consuming process of rubbing the powder onto the nail with a sponge-like applicator. The powder generally comes in a 1.5 g or otherwise small container and can easily be spilled onto surfaces and clothing. After applying the powder, a top coat is applied which seals the powder onto the nail but substantially dulls the shiny metallic effect of the powder. Another way to achieve the desired look is to apply a chrome nail polish that may produce a shiny metallic finish, but which shine lasts only a few hours.

As an alternative to nail polish, gel polish may be applied. Gel polish is typically preferred over regular nail polish as it is generally not subject to cracking, peeling or chipping and lasts days or weeks longer than regular nail polish. Gel polish and the fumes it expels into the air lack toxins found in regular nail polish, resulting in a more eco-friendly product. Rather than drying through solvent evaporation, gel polish is exposed to a UV or LED light wherein monomers undergo polymerization and harden—a process known as “curing.” The desired shiny, metallic finish may be achieved by applying chrome powder on an underlying coat of gel polish, followed by a gel top coat. Shiny metallic regular nail polish may also be applied to a gel polish base coat followed by a gel top coat, but the top coat may substantially reduce the shiny effect.

For the foregoing reasons, what is needed is a gel polish lacking toxic airborne chemicals found in regular nail polish such as formaldehyde, toluene, and triphenyl phosphate that provides a shiny, metallic effect and/or without the need of applying a chrome powder and/or lasting the duration of application to the nail and/or which does not dull with application of a top coat.

### SUMMARY

Disclosed herein is a chemical composition that satisfies the need for a gel nail polish with a shiny metallic effect free from toxic airborne chemicals.

In certain embodiments, the composition comprises monomers, aluminum compound, epoxy acrylate oligomer, an acrylic acid polymer and a photoinitiator. The chemicals may be mixed at room temperature. In certain embodiments, about 72% weight of the total composition is monomers, about 16% weight is aluminum compound, about 5% by weight is an epoxy acrylate oligomer, about 4% weight is an acrylic acid polymer and/or about 3% weight is a photoinitiator.

In a preferred embodiment, two monomers may be used. The monomers are known in the art as “film forming ingredients” forming a hard and glossy film on the exposed surface of the nail. They are consumed within seconds to minutes during the polymerization process taking place by exposing the polish to a UV or LED light. The first monomer is a methacrylate ester monomer and may be, for example, 2-hydroxyethyl methacrylate commonly referred to as “HEMA” with a Chemical Abstracts Service (CAS) number of 868-77-9 at about 12% weight of the total composition. The second monomer is a carbamate ester monomer and may be, for example, trimethylhexyl dicarbamate commonly referred to as “Di-HEMA” with a CAS number of 41137-60-4 at 60% weight of the total composition. In another embodiment only one monomer is used and may be, for example, trimethylhexyl dicarbamate or 2-hydroxyethyl methacrylate at 72% weight of the total composition.

The epoxy acrylate oligomer has characteristics such as adhesion and hardening, which makes it a desirable resin for UV or LED curing products. The oligomer may be, for example, bisphenol-A-epoxy diacrylate with a CAS number of 55818-57-0.

The acrylic acid polymer is acrylate polyurethane comprised of isophorone diisocyanate with a CAS number 4098-71-9 and polyester polyol polymer. The polyol polymer is comprised of adipic acid with a CAS number of 124-04-9 and 2-methyl-1, 2-popanediol with a CAS number of 558-43-0.

Photoinitiators absorb the wavelengths emitted from the UV or LED light and convert the wavelength into the energy needed to start the polymerization, or curing, process. The photoinitiator is 1-hydroxycyclohexyl phenyl ketone with a CAS number of 947-19-3. Alternatively, the photoinitiator may be benzophenone or a mixture of 1-hydroxycyclohexyl phenyl ketone and benzophenone.

Aluminum contained in the aluminum compound is responsible for the silver, metallic, mirror effect of the polish and is preferably 15.5-16.5% by weight of the total composition. The aluminum compound is preferably filtered prior to mixing with the remaining chemical of the metallic gel nail polish and is comprised of aluminum, stearic acid and a monomer.

A preferred method of applying the metallic gel nail polish may ensure the aluminum in the polish reflects off an applied top coat. The polish is preferably applied to fingernails and toenails, but may also be applied to solid surfaces. The preferred method of applying the metallic gel nail polish is first applying a standard base coat to the surface and curing the gel polish using standard curing practices. The next step is applying a coat of the metallic gel nail polish to the surface. Before curing, the user may wait about 10-60 seconds which gives time for the aluminum particles to evenly disperse across the surface due to surface tension differences between the aluminum and remainder of the chemical composition of the metallic gel nail polish, so that the shiny metallic effect is evenly distributed.

The next step is curing the metallic gel nail polish for about 2 minutes if using an about 36-watt UV light or for about 10-60 seconds if using an LED light. If applicable, another coat of the silver metallic chrome polish may be applied and cured to ensure the entire nail is covered with the polish. Optionally, a second coat of the base coat may be applied on top of the metallic gel nail polish and cured for about 2 minutes if using an about 36-watt UV light or for about 10-60 seconds if using an LED light to further protect and seal the silver metallic chrome polish. The next step is applying a top coat and curing for about 2 minutes if using a UV light or for about 10-60 seconds if using an LED light. If using a wipe-free top coat, the application process is complete. But if using a top coat not formulated as wipe-free, the last step is wiping the cured standard top coat preferably with a lint-free cloth to remove any sticky residue from the surface formed during the curing process.

Another method of applying the metallic gel nail polish is to apply a colored translucent gel polish over the metallic gel nail polish. This will result in a colored look (i.e. blue, purple, gold) with a shiny metallic effect. The first step is applying a base coat to the surface and curing the base coat for about 2 minutes if using an about 36-watt UV light or for about 10-60 seconds if using an LED light. The next step is applying a coat of the metallic gel nail polish to the surface. Before curing, the user should wait about 10-60 seconds so the aluminum particles may evenly disperse across the surface. The next step is curing the metallic gel nail polish for about 2 minutes seconds if using an about 36-watt UV light or for about 10-60 seconds if using an LED light. If desired, a second coat of the metallic gel nail polish may be applied and cured for about 2 minutes if using an about 36-watt UV light or for about 10-60 seconds if using an LED light.

The next step is to apply a colored translucent gel nail polish with a preferred translucency of about 83.5-84.5%. The translucent gel polish is preferably applied with one coat as to not reduce the translucency and permit the aluminum particles from the metallic gel nail polish to reflect off the translucent gel polish. The translucent gel polish is then cured for about 2 minutes if using an about 36-watt UV light or for about 10-60 seconds if using an LED light. The next step is applying a top coat to the surface and curing the standard clear top coat for about 2 minutes if using a UV light or for about 10-60 seconds if using an LED light. If using a wipe free top coat, the application process is complete. But if using a top coat not formulated as wipe-free, the last step is wiping the cured standard top coat preferably with a lint-free cloth.

As used herein, the terms below have the meanings indicated.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures, and rounding up decimals and fractions to the next whole number.

The term “nail polish” is a comprehensive term describing a nail polish composition, film, product (including products), system, kit, or the like, which is useful for providing, for example, aesthetic, therapeutic, or prophylactic benefits to the nail.

The term “base coat” as used herein, refers to the first coat applied and cured on the nail or other applied surface. Base coats are well known in the art and are formulated to protect the underlying nail or surface and, upon curing, are sticky to aid in optimal binding with the next applied coat applied. While base coats are not required, application is preferred.

The term “top coat” as used herein, refers to the exposed coat applied and cured on the nail or other applied surface. Top coats are well known in the art and preferably substantially clear, protecting the nail polish underneath and adding shine. Other top coats may include matte top coats which, when cured, give the underlying polish a matte, or non-shiny, look. And yet other top coats may have slight tints of color to give the underlying nail polish a colored tint respective to the top coat.

The term “standard curing practice” as used herein refers to the process of curing the gel polish by exposing the polish to a UV or LED light. The polish is cured by preferably exposing a polish-coated surface to an LED light for about 10-60 seconds, although more time may be necessary depending on the thickness of the gel polish. The polish may also cured by exposing the polish-coated surface to an about 36-Watt UV light, although about 10-48 Watt UV light may be used. When using the preferred about 36-Watt UV light, the polish will cure after being exposed to the light for about 2 minutes. Should higher or lower wattage be used, the time to cure will be more or less, respectively.

### BRIEF DESCRIPTION OF THE DRAWINGS

FIG. 1 illustrates a flowchart of the preferred method of applying the metallic gel nail polish.

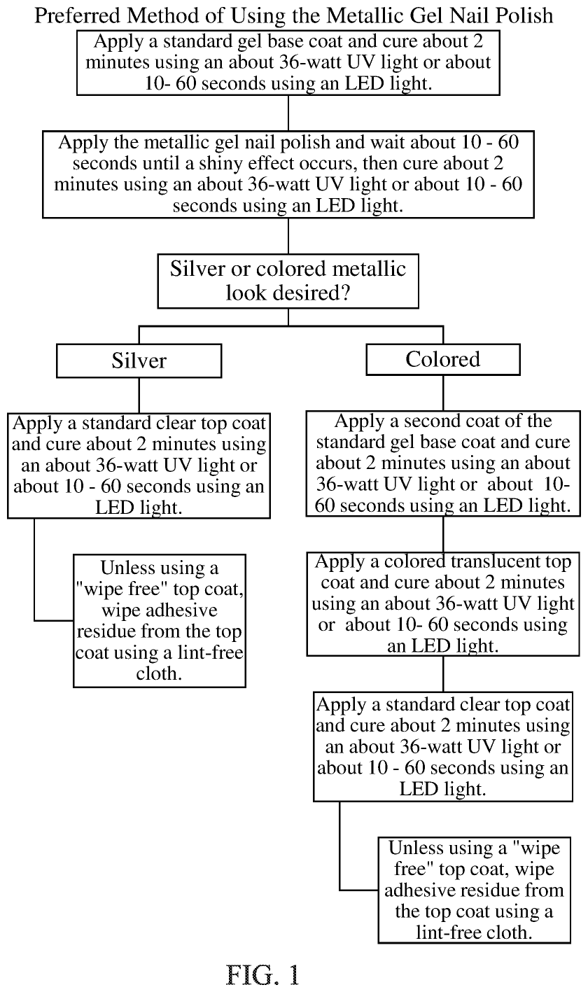

### DETAILED DESCRIPTION

The metallic gel nail polish of the preferred embodiment is generally comprised of monomers, aluminum compound, epoxy acrylate oligomer, acrylate polyurethane, and photoinitiator. The composition polymerizes upon exposure to a UV or LED light source and results in a shiny silver with a metallic effect achieved by order of applying the metallic gel polish such that aluminum particles of the aluminum compound reflect off a top coat.

In a preferred embodiment, the monomers are a methacrylate ester monomer, trimethylhexyl dicarbamate (Di-HEMA) (C₂₃H₃₈N₂O₈), at about 60% wt of the total composition and a carbamate ester monomer, 2-hydroxyethyl methacrylate (HEMA) (C₆H₁₀O₃), at about 12% wt of the total composition. The epoxy acrylate oligomer is preferably bisphenol A-epoxy diacrylate (C₂₁H₂₅ClO₅) at about 5% wt, the acrylate polyurethane is comprised of polyester polyol and isophorone diisocyanate (C₁₂H₁₈N₂O₂) at about 4% wt, and the photoinitiator is preferably free radical photoinitiator 1-hydroxycyclohexyl phenyl ketone (C₁₃H₁₆O₂) at about 3% wt. The polyester polyol is preferably comprised of adipic acid (C₆H₁₀O₄) and 2-methyl-1,2-propanediol (C₄H₁₀O₂).

The preferred embodiment also includes aluminum compound at about 16% wt of the total composition. While about 15.5% to 16.5% may be used, using less could decrease the shiny metallic effect of the polish, while adding more could increase production costs and lessen the ability of the metallic gel nail polish to cure. Solid aluminum is processed into a aluminum compound, a semi-solid aluminum pulp-like phase, by adsorbing a predetermined amount of stearic acid (CH₃(CH₂)₁₆COOH or C₁₈H₃₆O₂) onto solid aluminum. The adsorption process occurs by mixing powdered stearic acid, a monomer, and aluminum powder using a steel ball grinding machine for preferably 2-4 hours. The monomer is preferably 1-methoxy-2-propanol (C₄H₁₀O₂) and is about 85% wt, the stearic acid is about 3% wt, and the aluminum (Al) is about 12% wt of the composition. During the mixing process, stearic acid is adsorbed onto the aluminum powder through interaction of the stearic acid's carboxyl group with the aluminum. The mixture is kneaded using a kneading machine until a semi-solid, pulp results. The aluminum compound is then filtered by vacuum preferably using a 15μ filter to remove small impurities.

The metallic gel polish composition is made, for example, by mixing the preferably filtered aluminum compound with the monomer, epoxy acrylate oligomer, acrylate polyurethane, and photoinitiator mixture resulting in a final product with a preferred viscosity of about 200 cps at about 25° C.

### Example 1

| | Amount |
|---|---|
| Components | (wt %) |
| trimethylhexyl dicarbamate | 60 |
| aluminum compound | 16 |
| 2-hydroxyethyl methacrylate | 12 |
| bisphenol A-epoxy diacrylate | 5 |
| acrylate polyurethane | 4 |
| 1-hydroxycyclohexyl phenyl ketone | 3 |
| | 100 |

In another embodiment, the photoinitiator may be 2-hydroxy-2-methyl-1-phenyl-propane-1-one. In yet another embodiment, the photoinitiator may be benzophenone (C₁₃H₁₀O) or a mixture of 1-hydroxycyclohexyl phenyl ketone and benzophenone.

### Example 2

| | Amount |
|---|---|
| Components | (wt %) |
| trimethylhexyl dicarbamate | 60 |
| aluminum compound | 16 |
| 2-hydroxyethyl methacrylate | 12 |
| bisphenol A-epoxy diacrylate | 5 |
| acrylate polyurethane | 4 |
| 2-hydroxy-2-methyl-1-phenyl-propane-1-one | 3 |
| | 100 |

### Example 3

| | Amount |
|---|---|
| Components | (wt %) |
| trimethylhexyl dicarbamate | 60 |
| aluminum compound | 16 |
| 2-hydroxyethyl methacrylate | 12 |
| bisphenol A-epoxy diacrylate | 5 |
| acrylate polyurethane | 4 |
| benzophenone | 3 |
| | 100 |

### Example 4

| | Amount |
|---|---|
| Components | (wt %) |
| trimethylhexyl dicarbamate | 60 |
| aluminum compound | 16 |
| 2-hydroxyethyl methacrylate | 12 |
| bisphenol A-epoxy diacrylate | 5 |
| acrylate polyurethane | 4 |
| 1-hydroxycyclohexyl phenyl ketone/benzophenone | 3 |
| | 100 |

The monomers of the aforementioned embodiments may be only one monomer, trimethylhexyl dicarbamate or 2-hydroxyethyl methacrylate, at 72% weight of the total composition.

The following additional embodiments are provided:

### Embodiment 1

A metallic gel nail polish comprising: about 72% weight monomers; about 16% by weight aluminum compound; about 5% weight an epoxy acrylate oligomer; about 4% weight an acrylic acid polymer; and about 3% weight a photoinitiator.

### Embodiment 2

The aluminum compound of Embodiment 1 wherein the aluminum compound is comprised of about 12% by weight aluminum, about 3% by weight stearic acid, and about 85% by weight monomer.

### Embodiment 3

The monomer of any of Embodiments 1-2 wherein the monomer is 1-methoxy-2-propanol.

### Embodiment 4

The metallic gel nail polish of any of Embodiments 1-3 wherein the monomers are about 60% by weight trimethylhexyl dicarbamate (Di-HEMA) and 12% by weight 2-hydroxyethyl methacrylate (HEMA).

The shiny metallic mirror-like effect may also be achieved with regular nail polish by mixing the aluminum compound with a preferably clear-colored nail polish such that the aluminum compound is about 16% wt of the total regular nail polish composition. The regular nail polish may also be substantially clear-colored or may contain a slight tint of color.

The preferred method of application of the metallic gel nail polish includes chronologically applying and curing a first base coat, metallic gel nail polish, a second base coat and a top coat preferably to a fingernail or toenail. Optionally, a colored translucent gel polish may be applied between the metallic gel nail polish and top coat.

The first step of application is preferably coating the nail with a first base coat and curing using standard curing practices. Next, the metallic gel nail polish is preferably applied atop the base coat, followed by a waiting period of about 10-60 seconds or more until a shiny effect occurs. During the about 10-60-second time frame, the aluminum from the aluminum compound uniformly disperses across the nail through the differences of surface tension between the aluminum and metallic gel nail polish. To ensure uniformity of aluminum during application, the metallic gel nail polish should be shaken prior to use. After the shiny effect occurs, the metallic gel nail polish is cured using standard curing practices. Should a silver metallic look be desired, the final step of application is applying a top coat and curing using standard curing practices.

The next step is preferably applying a second base coat and curing using standard curing practices. Alternatively, the second base coat is not applied.

If a different colored shiny metallic look is desired, the next step is applying a colored translucent top coat and curing using standard curing practices. The translucent gel polish may be a variety of colors with a preferred translucency of about 84% to ensure the aluminum particles from the metallic gel nail polish reflect off the translucent gel polish to provide a colored shiny metallic effect. For example, applying a translucent yellow coat atop the metallic gel nail polish will produce a gold metallic color. The translucency of the gel polish may also be about 82-86% and still obtain an optimal shiny effect. The colored translucent gel polish is then cured using standard curing practices.

The next step is applying and curing a top coat using standard curing practices. A final step may be wiping the adhesive residue from the top coat preferably with a lint-free cloth if the top coat is not formulated as “wipe-free.”

The foregoing descriptions of embodiments have been presented only for purposes of illustration and description. They are not intended to be exhaustive or to limit the present invention to the forms disclosed. Accordingly, many modifications and variations will be apparent to practitioners skilled in the art. Additionally, the above disclosure is not intended to limit the present invention. The scope of the present invention is defined by the appended claims.

Insofar as the description above and the accompanying drawings disclose any additional subject matter that is not within the scope of the claims below, the inventions are not dedicated to the public and the right to file one or more applications to claim such additional inventions is reserved.

1. A metallic gel nail polish comprising:
at least a first monomer;
a semi-solid aluminum compound including stearic acid adsorbed on aluminum powder and a second monomer, the second monomer being different from said first monomer;
an epoxy acrylate oligomer;
an acrylic acid polymer; and
a photoinitiator.

2. A metallic gel nail polish comprising:
about 72% by weight at least a first monomer;
about 16% by weight an aluminum compound including stearic acid adsorbed on aluminum powder and a second monomer, the second monomer being different from said first monomer;
about 5% by weight an epoxy acrylate oligomer;
about 4% by weight an acrylic acid polymer; and
about 3% by weight a photoinitiator.

3. The metallic gel nail polish of claim 2 wherein the aluminum compound is comprised of about 12% weight aluminum, about 3% weight stearic acid and about 85% weight second monomer.

4. The metallic gel nail polish of claim 3 wherein the second monomer is 1-methoxy-2-propanol.

5. The metallic gel nail polish of claim 2 wherein the at least a first monomer is one of Di-HEMA trimethylhexyl dicarbamate or 2-hydroxyethyl methacrylate (HEMA).

6. The metallic gel nail polish of claim 2 wherein the at least a first monomer comprises two monomers including about 60% by weight Di-HEMA trimethylhexyl dicarbamate and 12% by weight 2-hydroxyethyl methacrylate (HEMA).

7. The metallic gel nail polish of claim 2 wherein the epoxy acrylate oligomer is bisphenol-A-epoxy diacrylate.

8. The metallic gel nail polish of claim 2 wherein the acrylic acid polymer is acrylate polyurethane.

9. The metallic gel nail polish of claim 2 wherein the photoinitiator is 1-hydroxycyclohexyl phenyl ketone.

10. The metallic gel nail polish of claim 2 wherein the photoinitiator is an alpha-hydroxylalkyl phenyl ketone.

11. The metallic gel nail polish of claim 2 wherein the photoinitiator is benzophenone.

12. The metallic gel nail polish of claim 2 wherein the photoinitiator is a mixture of 1-hydroxycyclohexyl phenyl ketone and benzophenone.

13. The metallic gel nail polish of claim 2 wherein the percentage of aluminum compound is between 15.5-16.5% by weight of the total composition.

14-25. (canceled)
